(19) United States
(12) Patent Application Publication
(10) Pub. No.: US 2016/0058957 A1
(43) Pub. Date: Mar. 3, 2016

(54) Method, system and device for detection of blood vessel penetration or prevention of blood vessel penetration for safely injecting liquids or gels under the skin surface

(71) Applicants: Lea Brodsky, Petah Tikva (IL); Isaac Zukier, Rosh Ha-Ayin (IL)

(72) Inventors: Lea Brodsky, Petah Tikva (IL); Isaac Zukier, Rosh Ha-Ayin (IL)

(21) Appl. No.: 14/839,210

(22) Filed: Aug. 28, 2015

Related U.S. Application Data
(60) Provisional application No. 62/044,277, filed on Aug. 31, 2014.
(60) Provisional application No. 62/088,637, filed on Dec. 7, 2014.

(51) Int. Cl. A61M 5/48; A61M 5/46

(52) U.S. Cl. CPC A61M 5/482; A61M 5/46

(57) ABSTRACT

Method, system and device for blood vessel penetration prevention or blood vessel penetration detection for safe injection of liquids or gels underneath the human skin surface is disclosed, either for wrinkle treatment or for other purposes.

Said method, system and device allow detection of needle contact with a blood vessel, detection of blood vessel penetration and detection of needle condition during its insertion into the patient's skin prior to injecting liquid or gel.

Said method, system and device allow prevention of blood vessel penetration by a needle, allow prevention of injection into a blood vessel and allow detection of defective needle.

Said device includes force sensor that measures said needle's insertion force required to overcome patient's tissues resistance to needle penetration, a controller, which performs force measurement analysis and positioning element, which stabilizes said needle in relation to the patient's skin without pressing the skin layers in the injection site.

### RELATED APPLICATIONS

The present application claims priority to U.S. provisional patent applications: 62/044,277 from Aug. 31, 2014 and 62/088,637 from Dec. 7, 2014 whose contents are included herein by reference.

### FIELD OF THE INVENTION

This invention relates to prevention of injury of blood vessels or prevention of injecting liquids or gels into a blood vessel. The method and device are based on analysis of insertion force measurement required to overcome patient's tissues resistance to the needle penetration during needle penetration under the skin surface.

### BACKGROUND OF THE INVENTION

Many aesthetic and medical procedures involve the injection of substances into the human body. Often, it is important to avoid the injection of said substances into blood vessels. For instance, when injecting hyaluronic acid dermal fillers, injection into blood vessels should be avoided since said blood vessel might be obstructed due to the high viscosity injected substance, resulting in tissue necrosis. Furthermore, while injecting, it is desirable not to injure or puncture blood vessels since it may cause bruising.

It is a goal of this invention to detect blood vessels during automated injection process, avoiding injuries or puncture of blood vessels and avoiding unintentional injection of substances into blood vessels. This goal is achieved by using at least one force sensor reactive to the needle or cannula insertion force linked to a controller, which reacts to the measured force profile.

Patent application WO 2014028285 A1 teaches a Manual device for inserting a catheter or needle into a blood vessel based on mechanically pushing forward a needle or catheter from its inside for as long as the insertion force is high. When the insertion force is low, the internal apparatus disengages due to the contraction of an element made of interlaced flexible elements and the catheter or the needle is no further introduced. This operation principle is intended for injecting a substance into a blood vessel only, and it cannot be used for avoiding injuring or puncturing a blood vessel, nor for avoiding the undesired injection of a substance into a blood vessel.

Many catheter, needle or cannula devices have been suited with force or pressure sensors for diverse purposes. Patent application WO 2006116997 A1 and U.S. Pat. No. 8,603,046, U.S. Pat. No. 8,618,948 and U.S. Pat. No. 8,937,553 disclose injectors with the ability of detecting malfunctions or problems such as blockage or occlusion of the syringe or the needle, or needle disengagement.

Some patents disclose devices with integrated force or pressure sensors located within or around the needle such as U.S. Pat. No. 3,550,583, which relates to a pressure sensor embedded within a needle, enabling the blood measurement within a blood vessel for example. U.S. Pat. No. 4,356,826 discloses a device with pressure sensors around the stabbing device tip, for sensing the tissue lateral pressure comprising: “a stabbing apparatus body having a sharp edge portion for penetrating the living body; sensor means disposed in said stabbing apparatus body for sensing a pressure to which said sensor means is subjected during penetration of a living body wall, and providing a pressure signal corresponding to said pressure; accumulator means coupled to said sensor means for accumulating information corresponding to the signal level of said pressure signal to provide an accumulated signal which corresponds to the depth of said penetration; and indicator means coupled to said accumulator means for indicating said penetration depth according to the magnitude of said accumulated signal.” This patent drawback consists on the need for sensors mounted in the needle tip, which are hard to manufacture and must be kept sterile prior to their utilization. Furthermore, since the sensors surround the needle, they can't sense the needle's tip contact with a blood vessel, so injury to blood vessels can't be avoided.

U.S. Pat. No. 6,221,023 teaches a device with a sensor mounted on the distal end of an intra-corporeal catheter, which detects pressure applied by surrounding tissue. The limitations and drawbacks of this patent are similar to those of U.S. Pat. No. 3,550,583.

U.S. Pat. No. 6,546,787 teaches a method of detecting at least one margin of a tissue structure of interest, comprising: a) providing a needle having a wall and a distal tip, a strain gage being connected to the needle wall at two spaced-apart locations, the strain gage generating a strain signal in response to strain on the wall of the needle; b) inserting the distal tip of the needle into a patient's tissue and advancing the needle distally into the tissue; c) monitoring the strain signal generated during said distal advancement of the needle; and d) analyzing the strain signal to detect the margin of the tissue structure. The drawback of this patent consists on the need for sensors mounted in the needle's tip, which are hard to manufacture and must be kept sterile prior to their utilization. Furthermore, since the sensors surround the needle, they can't sense the needle's tip contact with a blood vessel, so injury to blood vessels can't be avoided.

Patents WO 2006120619, WO 2008081438 and US 20100030111 refer to automated puncture systems or injectors intended for injecting into blood vessels or for taking blood samples from blood vessels. They are based on external, non-invasive sensors, which locate the blood vessel to be punctured, and a needle is inserted accordingly. These sensors may be based on near infrared imaging, optical coherence tomography, photo acoustic Imaging or ultrasound techniques, Doppler Effect sensors, pressure sensors, sensors that operate on radar principles, or optical sensors.

The drawback of these three patents is that they require very large or expensive additional equipment, and even with this equipment it will still be very difficult to automatically locate the blood vessels.

U.S. Pat. No. 5,314,410 and US 20150112261 teach devices based on pressure transmitted from the needle's tip through the fluid within the needle's lumen, while the fluid pressure sensing is based on a membrane. These two patents refer to devices, which are intended for manual operation. They might be able to detect or indicate the needle penetration of a blood vessel provided: (a) the needle has already penetrated the blood vessel—hence prohibiting the possibility of avoiding injury to the blood vessel, and (b) these patents do not refer to needles containing highly viscous substances such as hyaluronic acid, which would prevent pressure measurement.

U.S. Pat. No. 5,954,701, U.S. Pat. No. 7,449,008, U.S. Pat. No. 7,740,612, U.S. Pat. No. 8,608,665, U.S. Pat. No. 8,814,807, U.S. Pat. No. 8,926,525, US 20110046477 and US 20110202012 are based on a sensor for measuring the fluid pressure as transmitted through the needle's lumen. These patents refer to devices which might be able to detect the needle penetration of a blood vessel provided: (a) the needle has already penetrated the blood vessel—hence prohibiting the possibility of avoiding injury to the blood vessel, and (b) the needle does not contain a highly viscous substance such as hyaluronic acid, which would prevent pressure measurement.

### SUMMARY OF THE INVENTION

According to this invention there is provided a method, system and device for detection of blood vessel penetration or prevention of blood vessel penetration for safely injecting of liquids or gels under the skin's surface.

The device allows detection of blood vessel penetration, thus preventing injection of liquid or gel into the blood vessel during procedure for wrinkle treatment or for other purposes.

The device allows detection of blood vessel and prevention of blood vessel penetration by a needle, thus preventing blood vessels injury during injection of liquid or gel under the skin surface either for wrinkle treatment or for other purposes. The device may reduce the risk of injecting high viscosity substances adjacent to a blood vessel, thus avoiding its pressing and blockage by the injected substance pressure, preventing side effects like necrosis or blindness.

The device allows detection of bad needle's condition (dull, bent or defective needle), thus preventing patient's pain during injection of liquid or gel under the skin surface either for wrinkle treatment or for other purposes.

Said device includes a positioning element placed on the patient's skin. Said positioning element stabilizes the device in relation to the patient's skin surface and does not press skin layers in the injection site.

Said device includes a force sensor that allows measurement of said needle's insertion force required to overcome the patient's tissues resistance to the needle penetration and a controller, linked to said force sensor which performs force measurement analysis for blood vessel penetration prevention, and/or for needle's condition detection and/or for blood vessel penetration detection. In case blood vessel penetration event is detected, device controller will prevent the injection of said liquid or gel. In case needle's contact with a blood vessel is detected, said device controller will prevent the needle's penetration into the blood vessel, in case bad needle's condition is detected, said device controller will indicate the user about this condition.

The herein disclosed method can be used by the device to detect blood vessel penetration in order to prevent injection of liquid or gel into blood vessels (which may cause blindness or necrosis in case of hyaluronic acid dermal filler injections).

The herein disclosed method can be used by the device to detect said needle's contact with a blood vessel and prevent blood vessel's penetration in order to prevent bruising and undesired injection of liquid or gel into blood vessels (which may cause blindness or Necrosis in case of hyaluronic acid dermal filler injections). The herein disclosed method can be used by the device to detect needle's condition and to prevent patient's pain during the injection of liquid or gel.

Said method is based on measuring the needle's insertion force required to overcome the patient's tissues resistance to the needle penetration using the force sensor and a controller linked to said force sensor, which performs force measurement analysis for blood vessel detection and/or blood vessel penetration prevention. In case needle contact with a blood vessel event is detected, said device controller will prevent said needle's penetration into said blood vessel prior to the injection of liquid or gel. In case needle penetration of a blood vessel event is detected, said device controller will prevent the injection of liquid or gel.

The combination of said positioning element that stabilizes the device and the needle in relation to the patient's skin surface and does not press the patient's skin layers in the injection site and the force sensor measurement analysis allow precise detection of contact with the blood vessel and precise detection of blood vessel penetration.

Said positioning element allows the practitioner to position said device on the patient's skin, stabilizing said device in relation to the patient's skin surface during the injection process.

Said positioning element is designed in such manner that it may press the patient's skin layers in an area nearby or surrounding the injection site, but not in the injection site itself or too close to it.

Said positioning element may be designed in such manner that it does not press the patient's skin layers in the treated area, allowing the practitioner to see the injection site and the treated area clearly. Said positioning element may be designed in such manner that it may be placed in contact with the patient's skin, stabilizing the needle in parallel to the patient's skin surface, or perpendicular to the skin surface or at a desired angle relative to the patient's skin surface.

In one embodiment of this invention the device's purpose is to inject one of the currently available dermal fillers for wrinkle treatment avoiding penetration into the blood vessels under the patients' skin surface. In another embodiment of this invention, the device's purpose is to inject other liquids or gels avoiding penetration into the blood vessels under the patients' skin surface.

In one embodiment of this invention the device's purpose is to inject one of the currently available dermal fillers for wrinkle treatment avoiding injecting into the blood vessels under the patients' skin surface. In another embodiment of this invention, the device's purpose is to inject other liquids or gels avoiding injecting into the blood vessels under the patients' skin surface.

In one embodiment of this invention the device's purpose is to inject one of the currently available dermal fillers for wrinkle treatment while detecting needle's condition during its penetration under the patients' skin surface. In another embodiment of this invention, the device's purpose is to inject other liquids or gels while detecting needle's condition during its penetration under the patients' skin surface.

In one embodiment of this invention, the device includes a disposable transparent positioning element that is in contact with the patient's skin during the injection process. Said positioning element has a hole in its tip to allow said needle to move through said hole and to penetrate the patient's skin during the injection. As a result of said hole location (in the tip of said positioning element) and size, the patient's skin layers are not pressed in the needle penetration point, allowing precise blood vessel detection or blood vessel penetration detection.

In another embodiment of this invention, the device includes a positioning element that is in contact with the patient's skin during the injection process. Said positioning element may be disposable or not, may be transparent or not, and may be made of any suitable material and may have any suitable shape, as long as it is able to stabilize said device in relation to the patient's skin surface.

In one embodiment of this invention, said needle is attached to the tip of a syringe.

In another embodiment of this invention, a flexible tube links the syringe and the needle, and said needle is displaced independently of the syringe or container.

In one embodiment of this invention, said force sensor may be placed on top of the syringe barrel. In this embodiment said syringe barrel is pushed by an actuator during syringe displacement. In another embodiment of this invention, said force sensor may be placed on the top of the syringe's plunger, or it may be placed in any other suitable location that allows measuring said needle's insertion force required to overcome patient's tissues' resistance to said needle penetration.

In one embodiment of this invention said force sensor is connected to an electronic circuit, which functions as a device controller. Said force sensor's output signal is measured, read and analyzed by said device controller. According to said force sensor signal analysis said controller determines whether said needle's tip has touched a blood vessel wall or whether said needle has penetrated a blood vessel.

Said controller electronic circuit controls an actuator, which is responsible for syringe/needle displacement and it can optionally stop said syringe/needle displacement in case of contact with blood vessel detection or in case of blood vessel penetration detection. Said controller electronic circuit controls an actuator, which is responsible for syringe's plunger displacement, and in case a blood vessel has been detected or a blood vessel penetration event has been detected it will prevent said plunger displacement, thus preventing the injection of said liquid or gel.

In one embodiment of this invention said force sensor is connected to an electronic circuit, which functions as a device controller. Said force sensor's output signal is measured, read and analyzed by said device controller. According to the force sensor signal analysis said controller may determine whether the needle is not sharp, bent or defective, and may inform the practitioner accordingly.

In one embodiment of this invention, said actuator may be an electrical motor. In another embodiment of this invention, said actuator may be a pneumatic, hydraulic or any other suitable actuator able to push and displace said syringe or said needle.

In one embodiment of this invention, the syringe displacement motor may be an electrical motor, which, using a mechanical transmission, moves the syringe in a linear path.

In one embodiment of this invention, said motor may be a step motor. In another embodiment of this invention said linear motor may be a D.C. brush motor, a D.C. brushless motor, an AC motor, or any kind of electrical motor with an added motion, rotation or position detector or sensor for either directly or indirectly measuring said needle movement and for determination of needle's position. Said motion, rotation or position detector may be mechanical, electrical, optical or of any suitable kind

In one embodiment of this invention, the device may activate an audible alarm in case contact with a blood vessel event has been detected. In another embodiment of this invention, the device may activate an optical, vibratory or any kind of suitable alarm in case contact with a blood vessel event has been detected, in order to inform the practitioner of such event.

In one embodiment of this invention, the device may activate an audible alarm in case a blood vessel penetration event has been detected. In another embodiment of this invention, the device may activate an optical, vibratory or any kind of suitable alarm in case a blood vessel penetration event has been detected, in order to inform the practitioner of such event.

In one embodiment of this invention the needle is inserted in parallel to the patient's skin surface. In another embodiment of this invention the needle is inserted perpendicularly to the patient's skin surface, or it may be inserted at any suitable angle in relation to the patient's skin.

### BRIEF DESCRIPTION OF THE DRAWINGS

FIG. 1 shows the tip of the disposable positioning element and the needle (opaque)

FIG. 2 shows the tip of the disposable positioning element and the needle (transparent)

FIG. 3 shows a vertical central cutout of the disposable positioning element

FIG. 4 shows a vertical central cutout of the disposable positioning element placed on patient's skin, including the needle, the skin layers and a blood vessel

FIG. 5 shows the skin, the needle and a blood vessel position during six different stages of the injection process

FIG. 6 shows the device configuration

FIG. 7 shows an oscillogram of the force sensor measurement registered by an Oscilloscope during needle penetration of the skin surface. The oscillogram shows measured voltage as a function of time.

### LIST OF REFERENCE NUMERALS USED IN THE FIGURES

1. Disposable transparent positioning element
2. Disposable transparent positioning element's tip
3. Disposable transparent positioning element tip's center hole
4. Patient's skin surface
5. Pressed patient's skin layers
6. Not pressed patient's skin layers
7. Needle
8. Syringe
9. Force sensor
10. Syringe and force sensor holder
11. Piston of linear motor for syringe displacement
12. Linear motor for syringe displacement
13. Syringe plunger
14. Piston of linear motor for pushing the plunger
15. Linear motor for pushing the plunger
16. Device enclosure
17. Moment when needle tip touches the skin surface (measured by Force Sensor)
18. Moment when needle tip penetrates the skin (measured by Force Sensor)
19. Epidermis
20. Dermis
21. Device controller
22. Blood vessel
23. Moment when needle tip touches the blood vessel (measured by Force Sensor)
24. Moment when needle tip penetrates the blood vessel (measured by Force Sensor)

### DETAILED DESCRIPTION OF THE PREFERRED EMBODIMENTS

The present invention is described with reference to the accompanying drawings. According to this invention there is provided a method, system and device for detection of blood vessels or prevention of blood vessel penetration for safely injecting of liquids or gels under the skin surface.

The device allows prevention of blood vessel penetration, thus preventing blood vessels' injury (bruising) and/or allows detection of blood vessel penetration, thus preventing injection of liquid or gel into the blood vessel under the skin surface either for wrinkle treatment or for other purposes.

The drawings are schematic, not showing all the details such as the securing elements of the various items, for the sake of simplicity and clarity.

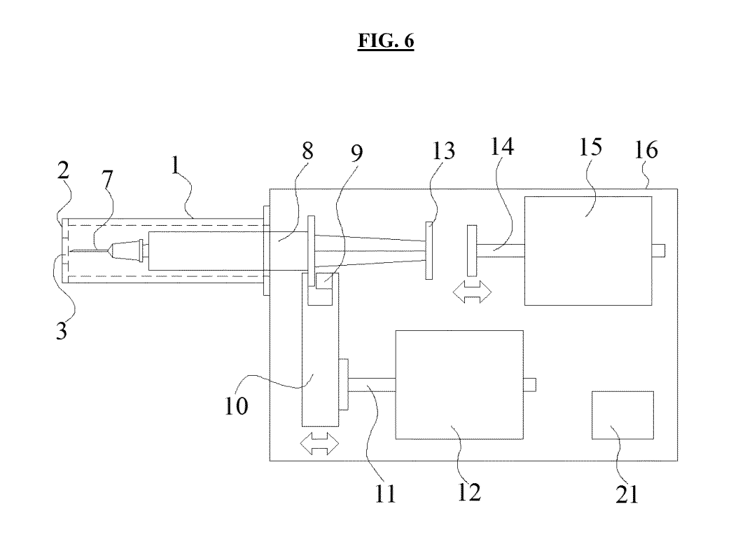

In the preferred embodiment of this invention the device enclosure 16 is designed for being hand-held by the operator, as shown in FIG. 6. The operator places the device on the patient's skin, just on top of the skin area to be treated, for instance a wrinkle The device should be placed on the treated skin area in such manner that the disposable transparent positioning element's tip 2 is in full contact with the area to be treated. The disposable transparent positioning element tip's center hole 3 should be placed precisely on the intended injection site.

A thin needle 7 is mounted on the tip of the syringe 8. In order to perform the injection, the linear motor for syringe displacement 12 and the syringe displacement linear motor's piston 11 are activated by the device controller 21. The activation of the linear motor for syringe displacement 12 and syringe displacement linear motor's piston 11 moves the syringe 8 and the needle 7 together either forward or backwards, allowing needle's insertion into patient's skin or its extraction from the patient's skin.

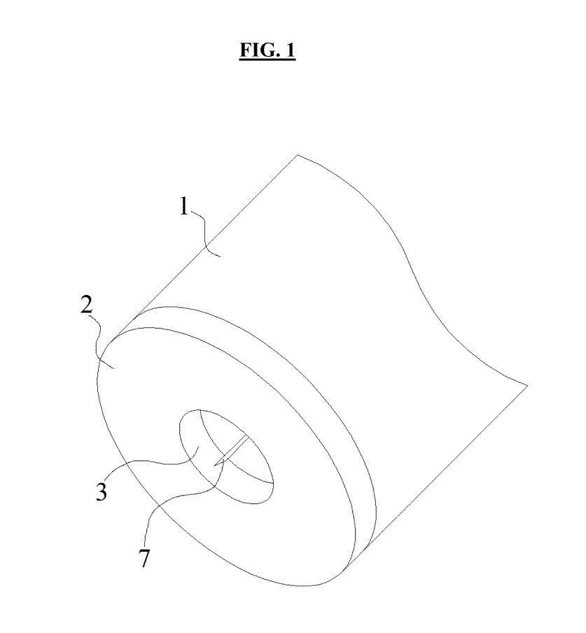

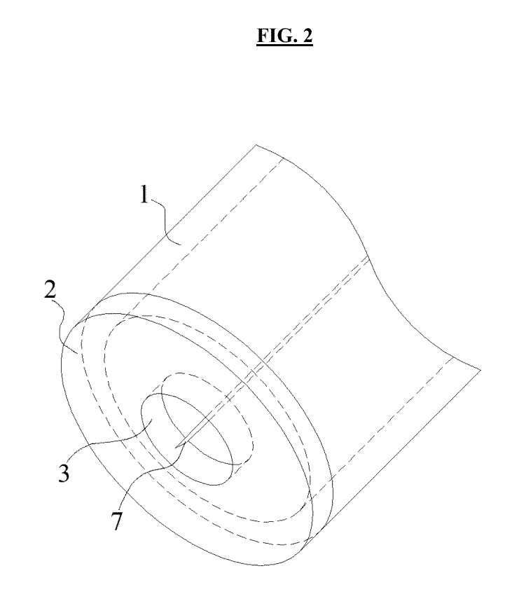

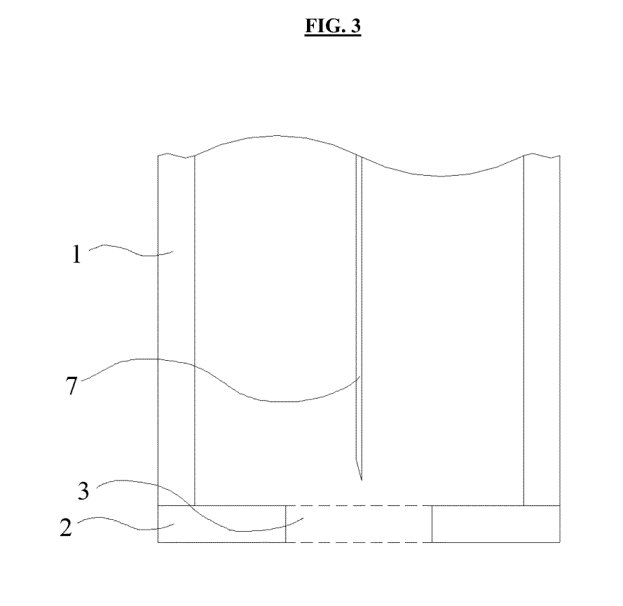

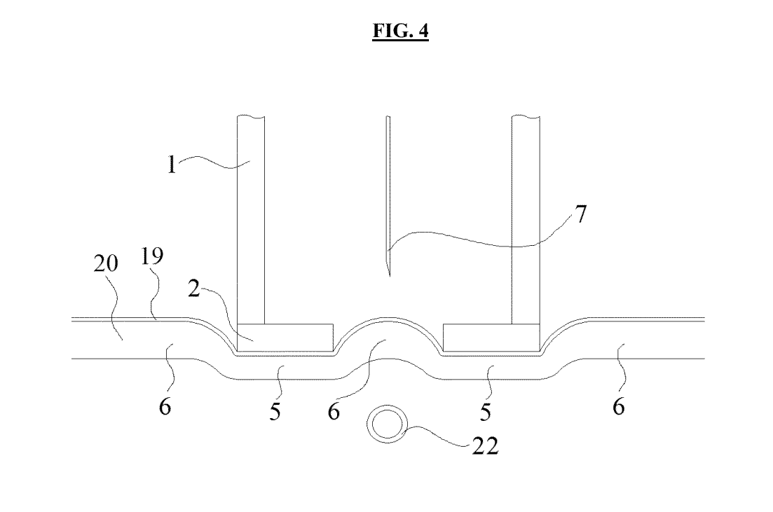

The disposable transparent positioning element 1 is shown in FIG. 1 as an opaque element, and in FIG. 2 as a transparent element. FIG. 3 shows a vertical central cutout of the disposable transparent element 1. The disposable transparent element 1 holds inside the syringe 8 with the needle 7 attached to the syringe's 8 tip. The disposable transparent positioning element 1 has a tube shape with a flat tip 2. Said flat tip 2 has a round hole 3 in its center, which allows to position the device on the patient's skin surface in such a manner that the device is completely stable and the skin layers (epidermis 19 and dermis 20) are not pressed in the injection site, allowing precise measurement of the needle's insertion force required to overcome the patient's tissues resistance to the needle 7 insertion. FIG. 4 depicts the skin layers of the patient and the blood vessel 22, showing the pressed skin layers 5 under the solid part of the disposable transparent positioning element's tip 2, and the not-pressed skin layers 6 in the central area of the disposable transparent positioning element's tip center hole 3. The external, thin skin layer is the epidermis 19, and under it the thicker dermis layer 20 is shown.

Force sensor 9 is placed on top of the syringe 8 barrel, which is pushed by the piston 11 of the linear motor for syringe displacement 12 during syringe displacement. Detection of event when needle 7 contacts blood vessel's 22 is determined by the needle's insertion force measured by the force sensor 9 during needle 7 insertion into the patient's skin. Device controller 21 receives output signal from the force sensor 9 and, accordingly, can stop the needle 7 insertion process.

Upon detection of needle's 7 contact with a blood vessel 22, device controller 21 immediately stops the operation of linear motor for syringe displacement 12, preventing any further insertion of the needle 7 into the blood vessel 22, thus preventing the blood vessel 22 penetration by the needle 7.

Device controller 21 may stop the operation of the linear motor for pushing the plunger 15, preventing the injection of liquid or gel adjacent to the blood vessel.

Detection of blood vessel penetration by the needle 7 is determined by the needle's 7 insertion force measured by the force sensor 9 during needle 7 insertion into the patient's skin. Device controller 21 receives output signal from the force sensor 9 and, accordingly, can stop the needle's 7 insertion process.

Device controller 21 stops the operation of the linear motor for pushing the plunger 15, preventing the injection of liquid or gel into the blood vessel.

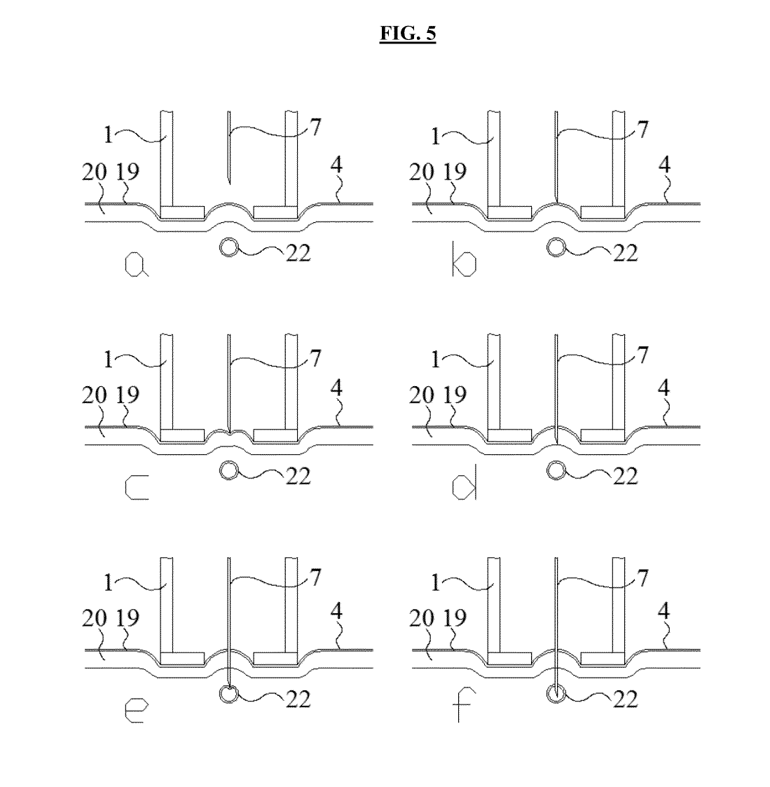

FIG. 5 shows the disposable transparent element 1, the needle 7, the patient's skin surface 4, the skin epidermis 19, the skin dermis 20 and the blood vessel 22 in six stages of the needle's 7 penetration prior to the injection process, including needle's 7 contact with blood vessel 22, and also needle's 7 penetration into a blood vessel 22.

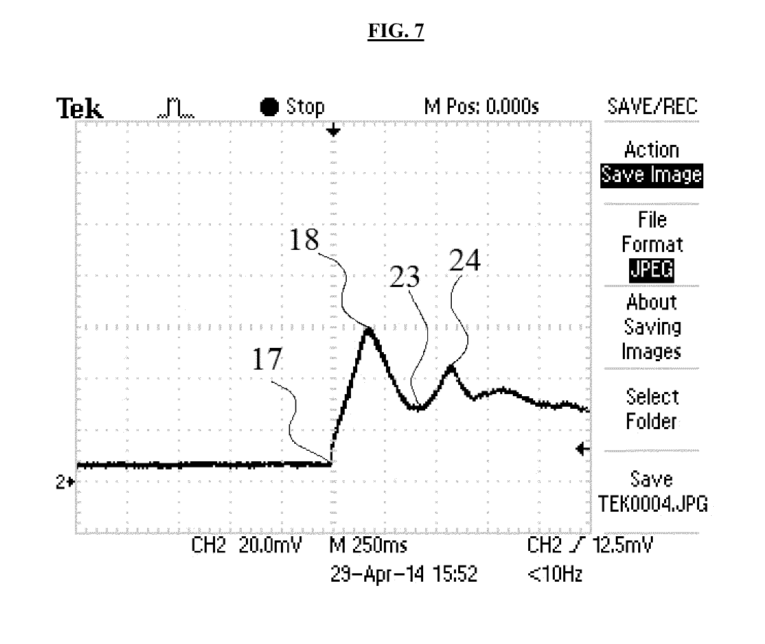

FIG. 7 shows an Oscillogram of the force sensor 9 signal recorded during needle's 7 penetration into the skin and penetration into a blood vessel 22, which this method and device prevents. The vertical axis reflects the force measured by the force sensor 9 and the horizontal axis reflects the time elapsed. Since the linear motor for syringe displacement 12 operates at constant speed, the horizontal axis represents syringe's 8 and needle's 7 displacement in a linear scale.

First, the needle 7 is at a certain distance from the skin surface 4 as shown in FIG. 5a. At this stage force sensor 9 senses only the frictional force of the advancing syringe 8 and the oscillogram shows a flat line.

As the needle 7 is displaced further toward the skin surface, it touches the epidermis 19 as shown in FIG. 5b. At this stage force sensor 9 starts sensing the force required to overcome the resistance of the epidermis 19 to needle's 7 tip insertion. Accordingly, the force measurement chart shows a steep increase in the sensed force, as shown in the inflexion point 17 in FIG. 7. As the needle 7 further pushes the skin layers down as depicted in FIG. 5c, the force measured by the force sensor 9 increases, as shown in FIG. 7 between points 17 and 18.

The needle 7 moves further forward, until the patient's skin is pierced as shown in FIG. 5d. The patient's skin penetration by the tip of the needle 7 happens at the moment 18 in FIG. 7. At this point the needle 7 insertion force required to overcome the skin resistance measured by force sensor 9 begins to decrease.

If the force measured at point 18 is higher than it should be when operating under normal conditions, then we can conclude that the needle's 7 condition is not good, either needle 7 is not sharp, or bent, or the needle 7 is defective.

In case the force measured at the peak point 18 increases by a predetermined amount or factor after a number of injections have been carried out, the device controller 21 activates a red LED (not shown), informing the practitioner that needle 7 is not sharp.

In case the force measured at the peak point 18 is higher than a predetermined threshold, the device controller 21 activates a yellow LED (not shown), informing the practitioner that needle 7 is defective.

The needle 7 moves deeper under the skin and the needle's 7 tip touches the blood vessel 22 wall as shown in FIG. 5e (point 23 in FIG. 7). At this point the needle 7 insertion force required to overcome the tissue resistance force measured by force sensor 9 begins to increase, as shown in FIG. 7 between points 23 and 24.

In order to prevent blood vessel's 22 penetration that would happen at point 24, the needle 7 insertion is stopped by device controller 21 somewhere between points 23 and 24 on FIG. 7, after it identified a raise in the force measured by force sensor 9. The syringe displacement linear motor's 12 operation is stopped by the device controller 21 when the needle's 7 tip touches and very slightly pushes the blood vessel 22 wall, thus preventing blood vessel 22 penetration.

The device controller 21 doesn't activate then the linear motor for pushing the plunger 15, preventing the injection of liquid or gel in case a blood vessel was detected. The device controller 21 activates the linear motor for syringe displacement 12 backwards, taking the syringe 8 back inside the disposable transparent positioning element 1.

The device controller 21 activates an on-board beeper (not shown), informing the practitioner that a blood vessel has been detected by the needle 7, and that the current injection process has been stopped.

In case the needle 7 has not touched a blood vessel, the syringe displacement linear motor's 12 operation is stopped by the device controller 21 when the desired penetration depth is achieved.

In order to prevent injection of liquid or gel into a blood vessel, assuming that following the stage depicted in FIG. 5e the needle 7 keeps moving forward, it penetrates the blood vessel's 22 wall, as shown in FIG. 5f. At this point the needle 7 insertion force required to overcome the blood vessel 22 resistance force measured by force sensor 9 peaks and begins to decrease, as shown in FIG. 7 point 24.

The device controller 21 detects said peak 24 shown in FIG. 7, which indicates the penetration of a blood vessel 22 by the needle 7.

The device controller 21 doesn't activate then the linear motor for pushing the plunger 15, preventing the injection of liquid or gel inside a blood vessel. The device controller 21 activates the linear motor for syringe displacement 12 backwards, taking the syringe 8 back inside the disposable transparent positioning element 1.

The device controller 21 then activates an on-board beeper (not shown), informing the practitioner that a blood vessel penetration event has occurred, and that the current injection process has been stopped.

1. A method and device for detecting or preventing blood vessel penetration for safely injecting of liquids or gels under the skins surface, said device comprising:
a container or a syringe containing liquid or gel connected to a needle for injecting said liquid or gel under the skins surface,
a force sensor that measures said needle's insertion force required to overcome patient's tissues resistance to the needle penetration,
a controller linked to said force sensor, which performs force measurement analysis for detecting or preventing blood vessel penetration, and
a positioning element, which stabilizes said needle in relation to the patient's skin.

2. The device according to claim 1, wherein needle insertion is done (performed) by a motor.

3. The device according to claim 1, wherein needle insertion is done (performed) by a pneumatic or hydraulic drive.

4. The device according to claim 1, wherein said needle insertion is stopped when a blood vessel is detected or penetrated.

5. The device according to claim 1, wherein an alarm is activated when a blood vessel is detected or penetrated.

6. The device according to claim 1, wherein needle insertion into the patient's skin is controlled by a micro-controller or microprocessor.

7. The device according to claim 1, where needle condition is detected, based on the force measurement analysis.

8. The device according to claim 1, wherein said positioning element is disposable.

9. The device according to claim 1, wherein said positioning element is transparent.

10. The device according to claim 1, wherein said positioning element stabilizes said device in relation to patient's skin surface without pressing skin layers at the injection site.

11. The device according to claim 1, wherein said needle is attached to the tip of said container or syringe.

12. The device according to claim 1, wherein a flexible tube connects said container or syringe to said needle.

13. The device according to claim 1, wherein said needle is moved independently of said container or syringe.

14. The device according to claim 1, wherein said force sensor is placed on top of said syringe barrel.

15. The device according to claim 1, wherein said force sensor is placed on top of said syringe's plunger.

16. The device according to claim 1, wherein said force sensor is placed in suitable location which allows measurement of said needle's insertion force required to overcome patient's tissues resistance to the needle penetration.
